SEQUENCE LISTING (5 sequences)

SEQ ID NO: 1 - protein, 348 aa, Fusarium heterosporum
MLLLSLLSAVTLAVASPVALEEYANSLEDRAVGVTSTDFTNFKFYIQHGAAAYCNSGTAAGAKITCSNNGCPTIESNGVTVVASFTGSKTGIGGYVSTDSSRKEIVVAIRGSSNIRNWLTNLDFDQSDCSLVSGCGVHSGFQNAWAEISAQASAAVAKARKANPSFKVVATGHSLGGAVATLSAANLRAAGTPVDIYTYGAPRVGNAALSAFISNQAGGEFRVTHDKDPVPRLPPLIFGYRHTTPEYWLSGGGGDKVDYAISDVKVCEGAANLMCNGGTLGLDIDAHLHYFQATDACNAGGFSWRRYRSAKRESIDMRATMTDAQLEAKLNSYVAMDQEYVKTHQNRT

SEQ ID NO: 2 - protein, 346 aa, Fusarium sp
MLLLPLLSAITLAVASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 3 - protein, 346 aa, Fusarium decemcellulare
MLFLSLLSAATLAVASPLADYVRALDDRAVSVSSQDYNNFKFYIQHGAAAYCNSETAAGQKITCGENSCPTVQSNGATVVASFVGSGTGIGGYVATDPTRKEIVLSFRGSSNIRNWLANLDFGQSDCTLTSNCGVHTGFQNAWNEVASKAKAAISSALKSNPTYKVIATGHSLGGAVATIGAANLRAGGTPVDIYTYGSPRVGNSELAAFISNQAGGEYRVTHGRDPVPRLPPLVFGYRHTSPEYWLSGGASDQNDYAITSIKTCEGGANLACNGGTLGLDIVAHLHYFQGTSDCNPGGIEWKRDTPAKRNKLVKRAEMTDAELEKKLNNYVQMDKEYVANHKDRS

SEQ ID NO: 4 - protein, 332 aa, Dactylonectria estremocensis
MLVLSLLSSVGVAAAASIGNYARILEDRAVTVTSANLANFKFYAQHSAAAYCNYGAKAGAKISCGGYCPTVESDGAVIVASFGGSATGIAGYVSTDATRKEIVISIRGSSNIRNWITNLDFGQDSCSSLVSGCGVHSGFQNAWDEIAAAAKAAVVSARTVNPSYVVVATGHSLGGAVATLAAAYLRKAGIPMDLYTFGSPRVGDGDFANFVTSQAGAEYRVTHEADPVPRLPPIIFGYRHTSPEYWLTGGSSDTTTYSVSSIKVCEGNANIGCNAGTLGLDIIAHLNYLQDISGCAAIGISWRRDELTDAELEAKINQYAKLDVEYVSYQPS

SEQ ID NO: 5 - protein, 177 aa, Metarhizium guizhouense
MKLNTTLLALATTALAAPASDVTTPKRQDINTVTDQLLFSSTLSQFEARRNAKEPPSLDWSSDGCTSSPDNPFGFPFLPACHRHDFGYQNYRIQKRFTKAAKAKIDSNFKTDLYYQCRSVSAKDACNGLADVYYEAVKEFGGGDATKRDRSDYDRAVAAYNAAVKEAQEQGLLPILD

(19) United States
(12) Patent Application Publication
(10) Pub. No.: US 2025/0008966 A1
(43) Pub. Date: Jan. 9, 2025

(54) IMPROVED ENZYMATIC MODIFICATION OF GALACTOLIPIDS IN FOOD

(71) Applicant: INTERNATIONAL N&H DENMARK APS, Kongens Lyngby (DK)

(72) Inventors: Svend Haaning, Stjaer (DK); Tina Lillan Jøergensen, Kongens Lyngby (DK); Lene Kragh, Kongens Lyngby (NL); Lone Broend Miller, Kongens Lyngby (CN); Jorn Borch Soe, Tilst (DK); Kefeng Ni, Shanghai (CN); Yuepeng Shang, Shanghai (CN); Xingxiang Xi, Shanghai (CN); Helong Hao, Shanghai (CN); Jens Frisbaek Sørensen, Aarhus N (DK)

(21) Appl. No.: 18/711,247

(22) PCT Filed: Nov. 17, 2022
(86) PCT No.: PCT/US2022/080062

§ 371 Date: May 17, 2024

(30) Foreign Application Priority Data
Nov. 17, 2021 (WO) PCT/CN2021/131128

(51) Int. Cl. A21D 8/04; A21D 10/00; A23K 20/189; A23L 15/00; C12N 9/18

(52) U.S. Cl. CPC A21D 8/042; A21D 10/002; A23K 20/189; A23L 15/25; C12N 9/18; C12Y 301/01026

(57) ABSTRACT

The present relates to lipases having galactolipase activity and no/low lyso-phospholipase activity. More particularly, the present invention relates to the use of such lipases for generation of emulsifying components from endogenous flour lipids.

### CROSS REFERENCE TO RELATED APPLICATIONS

This application claims priority to PCT Application No. PCT/CN2021/131128, filed Nov. 17, 2021, which is incorporated herein by reference in its entirety.

### TECHNICAL FIELD

The present invention relates to lipases and their use in the manufacture of food. The present invention further relates to methods of making dough and baked products using such lipases.

### BACKGROUND

The use of lipases to provide improved bread dough is well known. For example, in EP0585988 it is shown that the addition of a lipase to dough provided an anti-staling effect in bread baked therefrom. WO94/04035 teaches that an improved softness can be obtained by adding a lipase to dough. It has also been shown that exogenous lipases can modify bread volume.

While lipases have been described for their positive properties in the preparation of dough and baked products, the performance of prior art lipases has many drawbacks because prior art lipases have generally had multiple activities, reducing or eliminating the potential beneficial effect of the lipase. Therefore, there is a need in some food applications, particularly in baking, for improved lipases having higher specificity.

### SUMMARY OF THE INVENTION

In accordance with an aspect of the present invention, an isolated polypeptide is presented constituting a lipase having galactolipase activity of at least 100 DGDG-U per mg of the lipase and having a ratio of LPC-U/DGDG-U below 0.02. Optionally, the lipase has galactolipase activity of at least 150, 200, 250 or 300 DGDG-U per mg.

Optionally, the lipase has a ratio of LPC-U/DGDG-U below 0.01, 0.009, 0.007, 0.005, 0.003 or 0.001.

Optionally, the lipase is an engineered lipase.

In another aspect of the present invention, an isolated polypeptide is presented constituting a lipase having a galactolipase activity of at least 100 MGDG-U per mg protein of said lipase and having a ratio of MGDG-U/LPC-U above 70, 75, 80, 85, 90, 95 or 100.

In another aspect of the present invention, a nucleic acid sequence is presented encoding the isolated polypeptide as described above.

In another aspect of the present invention, a recombinant expression vector is presented having the polynucleotide as described above.

In another aspect of the present invention, a host cell is presented having the recombinant expression vector as described above. Optionally, the host cell is a bacterial, fungal, yeast, plant or mammalian cell.

In another aspect of the present invention, a method is presented of making a dough, the method having the step of admixing a dough component selected from the group consisting of flour, salt, water, sugar, fat, lecithin, oil and yeast with an isolated polypeptide as described above.

Optionally, the method includes adding at least one additional enzyme useful for improving dough and/or a baked product made therefrom. Optionally, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Optionally, the additional enzyme is an amylase which is optionally an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the phospholipase comprises SEQ ID NO: 5 or an active fragment thereof. Optionally, the active fragment is a mature polypeptide.

Optionally, the method includes adding an emulsifier. Optionally, the emulsifier is a phospholipid emulsifier. Optionally, the phospholipid emulsifier is lecithin or lyso-lecithin. Optionally, the emulsifier is a non-phospholipid emulsifier. Optionally, non-phospholipid emulsifier is DATEM, SSL, a monoglyceride or a diglyceride.

In another aspect of the present invention, a pre-mix for baking is presented having flour and an isolated polypeptide as described above.

Optionally, the pre-mix includes adding at least one additional enzyme useful for improving dough and/or a baked product made therefrom. Optionally, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Optionally, the additional enzyme is an amylase which is optionally an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the phospholipase comprises SEQ ID NO: 5 or an active fragment thereof. Optionally, the active fragment is a mature polypeptide.

In another aspect of the present invention, a method of creating a lyso-galactolipid in a lipid containing food matrix is presented having the step of adding to the lipid containing food matrix an isolated polypeptide as described above. Optionally, the lipid containing food matrix is selected from the group consisting of eggs and food products containing eggs, dough for sweet bakery goods, processed meat, milk based products, and vegetable oil.

In another aspect of the present invention, a method of creating a lyso-galactolipid in a lipid containing animal feed matrix or a grain based matrix is presented having the step of adding to the lipid containing to the lipid containing animal feed matrix or the grain based matrix an isolated polypeptide as described above.

In another aspect of the present invention, a dough is presented having an isolated polypeptide as described above. Optionally, the dough has improved dough extensibility and/or stability. Optionally, the dough has at least one additional enzyme useful for improving dough and/or a baked product made therefrom. Optionally, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Optionally, the additional enzyme is an amylase which is optionally an exoamylase. Optionally, the exoamylase is a maltogenic amylase. Optionally, the exoamylase is a non-maltogenic amylase. Optionally, the non-maltogenic amylase hydrolyses starch by cleaving off one or more linear malto-oligosaccharides, predominantly comprising from four to eight D-glucopyranosyl units, from the non-reducing ends of the side chains of amylopectin. Optionally, the additional enzyme is a phospholipase. Optionally, the phospholipase comprises SEQ ID NO: 5 or an active fragment thereof. Optionally, the active fragment is a mature polypeptide.

In another aspect of the present invention, a method of preparing a baked product is presented having the step of baking a dough as described above.

In another aspect of the present invention, a baked product is presented prepared according to the method as described above. Optionally, the baked product has at least one improved property selected from the group consisting of improved crumb pore size, improved uniformity of gas bubbles, no separation between crust and crumb, increased volume, increased crust crispiness and improved oven spring.

In another aspect of the present invention, an isolated polypeptide is presented constituting a lipase having at least 75, 80, 85, 90, 95, 98, 99 or 100% sequence identity to SEQ ID NO:3 or SEQ ID NO:4 or a lipase active fragment thereof. Optionally, the lipase active fragment is a mature polypeptide.

### BRIEF DESCRIPTION OF THE BIOLOGICAL SEQUENCES

SEQ ID NO: 1 sets forth the full length amino acid sequence of a lipase used in the commercial product Powerbake 4080.

SEQ ID NO: 2 sets forth the full length amino acid sequence of a lipase used in the commercial product Lipopan F.

SEQ ID NO:3 sets forth the full length amino acid sequence of CRC26230.

SEQ ID NO:4 sets forth the full length amino acid sequence of CRC26190.

SEQ ID NO:5 sets forth the full length amino acid sequence of the phospholipase (see also WO2019/121585 incorporated here by reference).

### BRIEF DESCRIPTION OF THE DRAWINGS

FIG. 1 depicts crusty roll volumes presented relative to volume of crusty roll with ‘SEQ ID NO:5’.

### DETAILED DESCRIPTION OF THE INVENTION

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Abbreviations

PL—phospholipid
LPL—lyso-phospholipid
GL—galactolipid
LGL—lyso-galactolipid
DGDG—di-galactosyl-di-glyceride
DGMG—di-galactosyl-mono-glyceride
MGDG—mono-galactosyl-di-glyceride
MGMG—mono-galactosyl-mono-glyceride
PC—phosphatidylcholine
LPC—lyso-phosphatidylcholine
PLA—phospholipase A
FFA—free fatty acid
PC-U—phospholipase activity
LPC-U—lyso-phospholipase activity
DGDG-U—galactolipase activity

### Definitions

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a galactolipase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptide, thereof, includes, but is not limited to, a culture broth containing secreted polypeptide expressed in a heterologous host cell.

The term “purified” refers to material (e.g., an isolated polypeptide or polynucleotide) that is in a relatively pure state, e.g., at least about 80% pure, at least about 90% pure, at least about 95% pure, at least about 98% pure, or even at least about 99% pure.

The term “enriched” refers to material (e.g., an isolated polypeptide or polynucleotide) that is in about 50% pure, at least about 60% pure, at least about 70% pure, or even at least about 70% pure.

A “pH range,” with reference to an enzyme, refers to the range of pH values under which the enzyme exhibits catalytic activity.

The terms “pH stable” and “pH stability,” with reference to an enzyme, relate to the ability of the enzyme to retain activity over a wide range of pH values for a predetermined period of time (e.g., 15 min., 30 min., 1 hour).

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded, and there may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

“Hybridization” refers to the process by which one strand of nucleic acid forms a duplex with, i.e., base pairs with, a complementary strand, as occurs during blot hybridization techniques and PCR techniques. Stringent hybridization conditions are exemplified by hybridization under the following conditions: 65° C. and 0.1×SSC (where 1×SSC=0.15 M NaCl, 0.015 M Na₃ citrate, pH 7.0). Hybridized, duplex nucleic acids are characterized by a melting temperature (Tₘ), where one half of the hybridized nucleic acids are unpaired with the complementary strand. Mismatched nucleotides within the duplex lower the Tₘ. Very stringent hybridization conditions involve 68° C. and 0.1×SSC.

A “synthetic” molecule is produced by in vitro chemical or enzymatic synthesis rather than by an organism.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., a galactolipase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein. Alternatively, specific activity can refer to the number of moles of product generated by an enzyme or enzyme preparation per unit of time under specific conditions.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty: OFF

Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

As used herein, the singular forms “a”, “an”, and “the” include both singular and plural referents unless the context clearly dictates otherwise.

The terms “comprising”, “comprises” and “comprised of as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +1-5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the term “lipase” refers to subset of Carboxylic Ester Hydrolases (EC 3.1.1) which catalyze the hydrolysis of ester bounds in lipids including triglycerides, phospholipids and galactolipids. It is known to those of skill in the art that lipases have a broad substrate range. Thus, for example, a phospholipase may also have activity on substrates other than phospholipids. For example, what is described as a phospholipase may be active on galactolipids. In this regard, a galactolipase may act on phospholipids. In general, the predominant activity of a lipase will be used to assign it to a class, e.g., galactolipases. Hence, a lipase of the instant invention may have activity towards different lipid moieties.

As used herein, the term “phospholipase” refers to an enzyme that hydrolyses phospholipids (including N-acyl-phosphatidylethanolamine) into fatty acids (saturated and/or unsaturated), lyso-phospholipids (including N-acyl-lyso-phosphatidylethanolamine), diacylgycerols, choline phosphate and phophatidates, depending on the site of hydrolysis. Phospholipases are further classified into types A, B, C and D.

As used herein, the term “phospholipase A” refers to enzymes that catalyze the hydrolysis of the ester bond of the fatty acid components of phospholipids. There are two different types of phospholipase A activity that can be distinguished. Phospholipase A1, as defined in enzyme entry EC 3.1.1.32, and phospholipase A2, as defined in enzyme entry EC 3.1.1.4, catalyze the deacylation of one fatty acyl group in the sn1 and sn2 positions, respectively, from a di-acyl-glycerophospholipid to produce lyso-phospholipid.

Another phospholipase is a “lyso-phospholipase” which catalyzes the hydrolysis of the fatty acyl group in the lyso-phospholipid.

As used herein, the term “galactolipase” refers to an enzyme that hydrolyses galactolipids into fatty acids (saturated and/or unsaturated), lyso-galactolipids and galactosylglycerols. Galactolipases catalyze the hydrolysis of galactolipids including di-galactosyl-di-glyceride (DGDG) and mono-galactosyl-di-glyceride (MGDG).

Another galactolipase is a “lyso-galactolipase” which catalyzes the hydrolysis of the fatty acyl group in the lyso-galactolipid.

As used herein, the phrase “a lyso-phospholipase/phospholipase activity ratio” or LPL/PL activity ratio’ means (LPC-U/mg protein)/(PC-U/mg protein) as set forth more fully below.

As used herein, the phrase “a lyso-phospholipase/galactolipase activity ratio” or LPL/GL activity ratio’ means (LPC-U/mg protein)/(DGDG-U/mg protein) as set forth more fully below.

As used herein, the phrase “engineered lipase” means a variant lipase obtained by altering a wildtype lipase through the use of protein engineering tools, known by a skilled person in the art. Many of the technologies are described in Protein Engineering Handbook, Vol I and II. WILEY-VCH Verlag GmbH & Co. KGaA. Ed. Stefan Lutz and Uwe T. Bornscheuer.

As used herein, the terms “phospholipids” or “phospholipid” or “PL” share the common features of fatty acids esterified to the 1 and 2 positions of the glycerol backbone with the phosphate group esterified to the 3 position.

As used herein, the terms “lyso-phospholipids”, “lyso-phospholipid” and “LPL” refer to a phospholipid containing one fatty acid esterified to the 1 or 2 position of the glycerol backbone with a phosphate group esterified to the 3 position.

As used herein, the term “galactolipids” or “galactolipid” or “GL” refer to esters of glycerol, fatty acids and sugar groups. The most common galactolipids are (but not limited to) mono-galactosyl-di-glyceride (MGDG) and di-galactosyl-di-glyceride (DGDG). These galactolipids share the common features of fatty acids (saturated and/or unsaturated) esterified to the 1 and 2 positions of the glycerol backbone with a sugar group linked to the 3 position.

As used herein, the terms “lyso-galactolipids”, “lyso-galactolipid”, and “LGL” refer to a galactolipid, containing one fatty acid esterified to the 1 or 2 position of the glycerol moiety and a sugar group linked to the 3 position.

As used herein, the phrase “lipid containing food matrix” refers to any food matrix that contains amounts of lipids.

As used herein, the phrase “a galactolipase/lyso-phospholipase activity ratio” or “GL/LPL activity ratio” means (MGDG-U/mg protein)/(LPC-U/mg protein) as set forth more fully below.

Other definitions are set forth below.

### Additional Mutations

In some embodiments, the lipases of the present invention include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at relatively high temperature. In some cases, the performance benefit may only be realized in the application e.g. bread making.

Furthermore, the lipases of the present invention may include any number of conservative amino acid substitutions. Exemplary conservative amino acid substitutions are listed in Table 1 below.

TABLE 1
Conservative Amino Acid Changes

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, |
| | | D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, D- |
| | | Met, Ile, D-Ile, Orn, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Trp, D-Trp, |
| | | Trans-3,4, or 5-phenylproline, cis-3,4, |
| | | or 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, Met(O), |
| | | D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Those of skill in the art will appreciate that some of the above-mentioned conservative mutations can be produced by genetic manipulation, while others are produced by introducing synthetic amino acids into a polypeptide by genetic or other means.

The lipases of the present invention may be “precursor,” “immature,” or “full-length,” in which case they include a signal sequence, or “mature,” in which case they lack a signal sequence and may be further truncated at the N- and/or C-terminus by proteolytic and/or non-proteolytic processing. In general, the mature forms of the polypeptides are generally the most useful. Unless otherwise noted, the amino acid residue numbering used herein refers to the mature forms of the respective lipase polypeptides. The present lipase polypeptides may also be truncated to remove the N or C-termini, so long as the resulting polypeptides retain lipase activity. In addition, lipase enzymes may be active fragments derived from a longer amino acid sequence. Active fragments are characterized by retaining some or all of the activity of the full-length enzyme but have deletions from the N-terminus, from the C-terminus or internally or combinations thereof.

The lipases of the present invention may be “chimeric” or “hybrid” polypeptides, in that they include at least a portion of a first lipase polypeptide, and at least a portion of a second lipase polypeptide. The lipases of the present invention may further include heterologous signal sequence, an epitope to allow tracking or purification, or the like. Exemplary heterologous signal sequences are from B. lichemformis amylase (LAT), B. subtilis (AmyE or AprE), T. reesei (AFP) and Streptomyces (CelA).

### Production of Enzymes

The enzymes of the present invention can be produced in host cells, for example, by secretion or intracellular expression. A cultured cell material (e.g., a whole-cell broth) having an enzyme can be obtained following secretion of the enzyme into the cell medium. Optionally, the enzyme can be isolated from the host cells, or even isolated from the cell broth, depending on the desired purity of the final enzyme. Suitable host cells include bacterial, fungal (including yeast and filamentous fungi), and plant cells (including algae). Mammalian cells can also be used to produce proteins according to the instant invention. Particularly useful host cells include Aspergillus niger, Aspergillus oryzae or Trichoderma reesei. Other host cells include bacterial cells, e.g., Bacillus subtilis or B. licheniformis, as well as Streptomyces, E. coli.

### Vectors

A DNA construct comprising a nucleic acid encoding an enzyme can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding galactolipase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding an enzyme can be transformed and replicated in a bacterial host cell as a means of propagating and amplifying the vector. The vector also may be transformed into an expression host, so that the encoding nucleic acids can be expressed as a functional galactolipase. Host cells that serve as expression hosts can include filamentous fungi, for example. The Fungal Genetics Stock Center (FGSC) Catalogue of Strains lists suitable vectors for expression in fungal host cells. See FGSC, Catalogue of Strains, University of Missouri, at www.fgsc.net (last modified Jan. 17, 2007). A representative vector is pJG153, a promoterless Cre expression vector that can be replicated in a bacterial host. See Harrison et al. (June 2011) Applied Environ. Microbiol. 77: 3916-22. pJG153 can be modified with routine skill to comprise and express a nucleic acid encoding a galactolipase.

A nucleic acid encoding an enzyme can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding a galactolipase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding an enzyme is expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from Trichoderma reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the galactolipase gene to be expressed or from a different Genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant galactolipase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of galactolipase for subsequent enrichment or purification. Extracellular secretion of galactolipase into the culture medium can also be used to make a cultured cell material comprising the isolated galactolipase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the galactolipase to a host cell organelle such as a peroxisome, or to a particular host cell compartment. Such a targeting sequence includes but is not limited to the sequence, SKL. For expression under the direction of control sequences, the nucleic acid sequence of the galactolipase is operably linked to the control sequences in proper manner with respect to expression.

The procedures used to ligate the DNA construct encoding a galactolipase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3^{rd} ed., 2001).

### Transformation and Culture of Host Cells

An isolated cell, either comprising a DNA construct or an expression vector, is advantageously used as a host cell in the recombinant production of an enzyme according to the instant invention. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species.

Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. An enzyme expressed by a fungal host cell can be glycosylated, i.e., will comprise a glycosyl moiety. The glycosylation pattern can be the same or different as present in the wild-type galactolipase. The type and/or degree of glycosylation may impart changes in enzymatic and/or biochemical properties.

It may be advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. Any gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding an enzyme is stably integrated into a host cell chromosome. Transformants are then selected and purified by known techniques.

The preparation of Trichoderma sp. for transformation, for example, may involve the preparation of protoplasts from fungal mycelia. See Campbell et al. (1989) Curr. Genet. 16: 53-56. The mycelia can be obtained from germinated vegetative spores. The mycelia are treated with an enzyme that digests the cell wall, resulting in protoplasts. The protoplasts are protected by the presence of an osmotic stabilizer in the suspending medium. These stabilizers include sorbitol, mannitol, potassium chloride, magnesium sulfate, and the like. Usually, the concentration of these stabilizers varies between 0.8 M and 1.2 M, e.g., a 1.2 M solution of sorbitol can be used in the suspension medium.

Uptake of DNA into the host Trichoderma sp. strain depends upon the calcium ion concentration. Generally, between about 10-50 mM CaCl₂ is used in an uptake solution. Additional suitable compounds include a buffering system, such as TE buffer (10 mM Tris, pH 7.4; 1 mM EDTA) or 10 mM MOPS, pH 6.0 and polyethylene glycol. The polyethylene glycol is believed to fuse the cell membranes, thus permitting the contents of the medium to be delivered into the cytoplasm of the Trichoderma sp. strain. This fusion frequently leaves multiple copies of the plasmid DNA integrated into the host chromosome.

Usually, transformation of Trichoderma sp. uses protoplasts or cells that have been subjected to a permeability treatment, typically at a density of 10⁵ to 10⁷/mL, particularly 2×10⁶/mL. A volume of 100 μL of these protoplasts or cells in an appropriate solution (e.g., 1.2 M sorbitol and 50 mM CaCl₂)) may be mixed with the desired DNA. Generally, a high concentration of PEG is added to the uptake solution. From 0.1 to 1 volume of 25% PEG 4000 can be added to the protoplast suspension; however, it is useful to add about 0.25 volumes to the protoplast suspension. Additives, such as dimethyl sulfoxide, heparin, spermidine, potassium chloride and the like, may also be added to the uptake solution to facilitate transformation. Similar procedures are available for other fungal host cells. See, e.g., U.S. Pat. No. 6,022,725.

As used herein, Protein Identification (“JGI PID”) numbers for native Trichoderma genes reference Version 2 of the Trichoderma reesei QM6a genome sequence assembly generated by the Department of Energy Joint Genome Institute (JGI). (The Genome Portal of the Department of Energy Joint Genome Institute, Grigoriev et al., Nucleic Acids Res 2012 January; 40 (Database issue):D26-32. doi: 10.1093/nar/gkr947). The JGI assembled Scaffold sequences and annotated genes have also been deposited in GeneBank (The National Center for Biotechnology) under the nucleotide accession numbers GL985056.1 through GL985132.1.

### Expression

A method of producing an enzyme of the instant invention may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of a galactolipase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of a galactolipase. Fermentation may, therefore, be understood as comprising shake flask cultivation, small- or large-scale fermentation (including continuous, batch, fed-batch, or solid-state fermentations) in laboratory or industrial fermenters performed in a suitable medium and under conditions allowing the galactolipase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding an enzyme in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of a galactolipase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

An expression host also can be cultured in the appropriate medium for the host, under aerobic conditions. Shaking or a combination of agitation and aeration can be provided, with production occurring at the appropriate temperature for that host, e.g., from about 25° C. to about 75° C. (e.g., 30° C. to 45° C.), depending on the needs of the host and production of the desired galactolipase. Culturing can occur from about 12 to about 100 hours or greater (and any hour value there between, e.g., from 24 to 72 hours). Typically, the culture broth is at a pH of about 4.0 to about 8.0, again depending on the culture conditions needed for the host relative to production of a galactolipase.

### Methods for Enriching and Purifying Enzymes

Fermentation, separation, and concentration techniques are well known in the art and conventional methods can be used in order to prepare an enzyme polypeptide-containing solution.

After fermentation, a fermentation broth is obtained, the microbial cells and various suspended solids, including residual raw fermentation materials, are removed by conventional separation techniques in order to obtain an enzyme solution. Filtration, centrifugation, microfiltration, rotary vacuum drum filtration, ultrafiltration, centrifugation followed by ultra-filtration, extraction, or chromatography, or the like, are generally used.

It is desirable to concentrate an enzyme polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

The enzyme solution is concentrated into a concentrated enzyme solution until the enzyme activity of the concentrated galactolipase polypeptide-containing solution is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides. Exemplary metal halides include sodium chloride, potassium chloride, sodium bromide, potassium bromide and blends of two or more of these metal halides. The metal halide precipitation agent, sodium chloride, can also be used as a preservative.

The metal halide precipitation agent is used in an amount effective to precipitate a galactolipase. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing.

Generally, at least about 5% w/v (weight/volume) to about 25% w/v of metal halide is added to the concentrated enzyme solution, and usually at least 8% w/v. Generally, no more than about 25% w/v of metal halide is added to the concentrated enzyme solution and usually no more than about 20% w/v. The optimal concentration of the metal halide precipitation agent will depend, among others, on the nature of the specific galactolipase polypeptide and on its concentration in the concentrated enzyme solution.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously.

Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. The organic compound precipitation agents can be, for example, linear or branched alkyl esters of 4-hydroxybenzoic acid, wherein the alkyl group contains from 1 to 10 carbon atoms, and blends of two or more of these organic compounds. Exemplary organic compounds are linear alkyl esters of 4-hydroxybenzoic acid, wherein the alkyl group contains from 1 to 6 carbon atoms, and blends of two or more of these organic compounds. Methyl esters of 4-hydroxybenzoic acid, propyl esters of 4-hydroxybenzoic acid, butyl ester of 4-hydroxybenzoic acid, ethyl ester of 4-hydroxybenzoic acid and blends of two or more of these organic compounds can also be used. Additional organic compounds also include but are not limited to 4-hydroxybenzoic acid methyl ester (named methyl PARABEN), 4-hydroxybenzoic acid propyl ester (named propyl PARABEN), which also are both preservative agents. For further descriptions, see, e.g., U.S. Pat. No. 5,281,526.

Addition of the organic compound precipitation agent provides the advantage of high flexibility of the precipitation conditions with respect to pH, temperature, galactolipase concentration, precipitation agent concentration, and time of incubation.

The organic compound precipitation agent is used in an amount effective to improve precipitation of the enzyme by means of the metal halide precipitation agent. The selection of at least an effective amount and an optimum amount of organic compound precipitation agent, as well as the conditions of the precipitation for maximum recovery including incubation time, pH, temperature and concentration of enzyme, will be readily apparent to one of ordinary skill in the art, in light of the present disclosure, after routine testing.

Generally, at least about 0.01% w/v of organic compound precipitation agent is added to the concentrated enzyme solution and usually at least about 0.02% w/v. Generally, no more than about 0.3% w/v of organic compound precipitation agent is added to the concentrated enzyme solution and usually no more than about 0.2% w/v.

The concentrated polypeptide solution, containing the metal halide precipitation agent, and the organic compound precipitation agent, can be adjusted to a pH, which will, of necessity, depend on the enzyme to be enriched or purified. Generally, the pH is adjusted at a level near the isoelectric point of the galactolipase. The pH can be adjusted at a pH in a range from about 2.5 pH units below the isoelectric point (pI) up to about 2.5 pH units above the isoelectric point.

The incubation time necessary to obtain an enriched or purified enzyme precipitate depends on the nature of the specific enzyme, the concentration of enzyme, and the specific precipitation agent(s) and its (their) concentration. Generally, the time effective to precipitate the enzyme is between about 1 to about 30 hours; usually it does not exceed about 25 hours. In the presence of the organic compound precipitation agent, the time of incubation can still be reduced to less about 10 hours and in most cases even about 6 hours.

Generally, the temperature during incubation is between about 4° C. and about 50° C. Usually, the method is carried out at a temperature between about 10° C. and about 45° C. (e.g., between about 20° C. and about 40° C.). The optimal temperature for inducing precipitation varies according to the solution conditions and the enzyme or precipitation agent(s) used.

The overall recovery of enriched or purified enzyme precipitate, and the efficiency with which the process is conducted, is improved by agitating the solution comprising the enzyme, the added metal halide and the added organic compound. The agitation step is done both during addition of the metal halide and the organic compound, and during the subsequent incubation period. Suitable agitation methods include mechanical stirring or shaking, vigorous aeration, or any similar technique.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

During fermentation, an enzyme polypeptide accumulates in the culture broth. For the isolation, enrichment, or purification of the desired galactolipase, the culture broth is centrifuged or filtered to eliminate cells, and the resulting cell-free liquid is used for enzyme enrichment or purification. In one embodiment, the cell-free broth is subjected to salting out using ammonium sulfate at about 70% saturation; the 70% saturation-precipitation fraction is then dissolved in a buffer and applied to a column such as a Sephadex G-100 column and eluted to recover the enzyme-active fraction. For further enrichment or purification, a conventional procedure such as ion exchange chromatography may be used.

Enriched or purified enzymes can be made into a final product that is either liquid (solution, slurry) or solid (granular, powder).

### DESCRIPTION OF THE PREFERRED EMBODIMENTS

In accordance with an aspect of the present invention, an isolated polypeptide is presented constituting a lipase having galactolipase activity of at least 100 DGDG-U per mg of the lipase and having a ratio of LPC-U/DGDG-U below 0.02. Preferably, the lipase has galactolipase activity of at least 150, 200, 250 or 300 DGDG-U per mg.

Without being bound by theory, the novel lipases of the present invention have a narrower substrate specificity than prior art lipases. The novel lipases of the instant invention produce less free fatty acid as compared with prior art baking lipases while maintaining very good baking performance. Controlled formation of free fatty acid on a lower level compared with prior art baking lipase is very important because free fatty acids are not preferred in food systems because they might give rise to oxidation and off-flavor formation

Preferably, the lipase has a ratio of LPC-U/DGDG-U below 0.01, 0.009, 0.007, 0.005, 0.003 or 0.001.

Preferably, the lipase is an engineered lipase.

In another aspect of the present invention, a nucleic acid sequence is presented encoding the isolated polypeptide as described above.

In another aspect of the present invention, a recombinant expression vector is presented having the polynucleotide as described above.

In another aspect of the present invention, a host cell is presented having the recombinant expression vector as described above. Preferably, the host cell is a bacterial, fungal, yeast, plant or mammalian cell.

In another aspect of the present invention, a method is presented of making a dough, the method having the step of admixing a dough component selected from the group consisting of flour, salt, water, sugar, fat, lecithin, oil and yeast with an isolated polypeptide as described above.

Preferably, the method includes adding at least one additional enzyme useful for improving dough and/or a baked product made therefrom. Preferably, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Preferably, the additional enzyme is an amylase which is preferably an exoamylase. Preferably, the exoamylase is a maltogenic amylase. Preferably, the exoamylase is a non-maltogenic amylase. Preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. Preferably, the phospholipase comprises SEQ ID NO: 5 or an active fragment thereof. Preferably, the active fragment is a mature polypeptide.

Preferably, the method includes adding an emulsifier. Preferably, the emulsifier is a phospholipid emulsifier. Preferably, the phospholipid emulsifier is lecithin or lyso-lecithin. Preferably, the emulsifier is a non-phospholipid emulsifier. Preferably, non-phospholipid emulsifier is DATEM, SSL, a monoglyceride or a diglyceride.

In another aspect of the present invention, a pre-mix for baking is presented having flour and an isolated polypeptide as described above.

Preferably, the pre-mix includes adding at least one additional enzyme useful for improving dough and/or a baked product made therefrom. Preferably, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Preferably, the additional enzyme is an amylase which is preferably an exoamylase. Preferably, the exoamylase is a maltogenic amylase. Preferably, the exoamylase is a non-maltogenic amylase. Preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. Preferably, the phospholipase comprises SEQ ID NO: 5 or an active fragment thereof. Preferably, the active fragment is a mature polypeptide.

In another aspect of the present invention, a method of creating a lyso-galactolipid in a lipid containing food matrix is presented having the step of adding to the lipid containing food matrix an isolated polypeptide as described above. Preferably, the lipid containing food matrix is selected from the group consisting of eggs and food products containing eggs, dough for sweet bakery goods, processed meat, milk based products, and vegetable oil.

In another aspect of the present invention, a method of creating a lyso-galactolipid in a lipid containing animal feed matrix or a grain based matrix is presented having the step of adding to the lipid containing to the lipid containing animal feed matrix or the grain based matrix an isolated polypeptide as described above.

In another aspect of the present invention, a dough is presented having an isolated polypeptide as described above. Preferably, the dough has improved dough extensibility and/or stability. Preferably, the dough has at least one additional enzyme useful for improving dough and/or a baked product made therefrom. Preferably, the additional enzyme is an amylase, cyclodextrin glucanotransferase, peptidase, transglutaminase, lipase other than said isolated polypeptide, phospholipase, galactolipase, cellulase, hemicellulase, protease, protein disulfide isomerase, glycosyltransferase, peroxidase, lipoxygenase, laccase, lactase, or oxidase. Preferably, the additional enzyme is an amylase which is preferably an exoamylase. Preferably, the exoamylase is a maltogenic amylase. Preferably, the exoamylase is a non-maltogenic amylase. Preferably, the non-maltogenic amylase hydrolyses starch by cleaving off one or more linear malto-oligosaccharides, predominantly comprising from four to eight D-glucopyranosyl units, from the non-reducing ends of the side chains of amylopectin. Preferably, the additional enzyme is a phospholipase. Preferably, the phospholipase comprises SEQ ID NO: 5 or an active fragment thereof. Preferably, the active fragment is a mature polypeptide.

In another aspect of the present invention, a method of preparing a baked product is presented having the step of baking a dough as described above.

In another aspect of the present invention, a baked product is presented prepared according to the method as described above. Preferably, the baked product has at least one improved property selected from the group consisting of improved crumb pore size, improved uniformity of gas bubbles, no separation between crust and crumb, increased volume, increased crust crispiness and improved oven spring.

In another aspect of the present invention, an isolated polypeptide is presented constituting a lipase having at least 75, 80, 85, 90, 95, 98, 99 or 100% sequence identity to SEQ ID NO:3 or SEQ ID NO:4 or a lipase active fragment thereof. Preferably, the lipase active fragment is a mature polypeptide.

In another aspect of the present invention, an isolated polypeptide is presented constituting a lipase having a galactolipase activity of at least 100 MGDG-U per mg protein of said lipase and having a ratio of MGDG-U/LPC-U above 70, 75, 80, 85, 90, 95 or 100. Without being bound by theory, it may be beneficial that a lipase used for baking have a high activity on MGDG and a low activity on LPC.

### EXAMPLES

### Assays and Methods

### Enzyme Characterization Assays

### PC-U Assay:

Phospholipase activity (PC-U) was determined using the following assay: A substrate solution was made by dissolving 1% (w/v) L-α Phosphatidylcholine Soy-95% (Avanti 441601G, Avanti Polar Lipids, USA), 6.5% (w/v) TRITON™-X 100 (Sigma X-100), and 5 mM CaCl₂) in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate (ThermoScientific #269620) and using a thermomixer (iEMS Incubator/shaker, Thermo Scientific). The assay was run as follows; 35 μL of substrate solution is mixed with 5 μL of enzyme sample and incubated for 600 sec at 30° C. The amount of free fatty acid liberated during enzymation was measured using the NEFA kit (WakoChemicals GmbH, Germany, NEFA-HR(2) R1 #434-91795 and NEFA-HR(2) R2 #436-91995).

The NEFA kit is composed of two reagents:

### NEFA-HR(2) R1:

50 mM Phosphate buffer pH 7.0 containing
0.53 U/mL Acyl-CoA Synthase (ACS)
0.31 mM coenzyme A (CoA)
4.3 mM adenosine 5-triphosphate disodium salt (ATP)
1.5 mM 4-amino-antipyrine (4-AA)
2.6 U/mL Ascorbate oxidase (AOD)
0.062% Sodium azide

### NEFA-HR(2) R2:

2.4 mM 3-Methyl-N-Ethyl-N-(E-Hydroxyethyl)-Aniline (MEHA)
12 U/mL Acyl-CoA oxidase (ACOD)
14 U/mL Peroxidase (POD)

After incubation the substrate-enzyme mixture is added 113 μL NEFA-HR(2) R1 and incubated for 300 sec at 37° C. Next, 56 μL NEFA-HR(2) R2 was added and the mixture was incubated for 300 sec at 37° C. Finally, OD 540 nm was measured (Molecular Devices, SpectraMaxPlus).

Enzyme activity was calculated based on the use of an enzyme standard of SEQ ID NO: 1. The enzyme standard was assigned an activity of 12174 PC-U/g based on the use of an oleic acid standard. Enzyme activity PC-U is calculated as micromole fatty acid produced per minute under assay conditions.

### LPC-U Assay:

Lysophospholipase activity (LPC-U) was determined using the following assay: A substrate solution was made by dissolving 1% 1-oleoyl-2-hydroxy-sn-Glycero-3-phosphocholine (Avanti 845875P, Avanti Polar lipid, USA), 6.5% (w/v) TRITON™-X 100 (Sigma X-100), and 5 mM CaCl2) in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate

ThermoScientific #269620) and using a thermomixer (iEMS Incubator/shaker, Thermo Scientific). The assay was run as follows; 35 μL of substrate solution is mixed with 5 μL of enzyme sample and incubated for 600 sec at 30° C.

The amount of free fatty acid liberated during enzymation was measured using the NEFA kit and enzyme activity was calculated based on the use of an enzyme standard of SEQ ID NO: 1. The enzyme standard was assigned an activity of 364 LPC-U/g based on the use of an oleic acid standard.

### DGDG-U Assay:

Galactolipase activity (DGDG-U) was determined using the following assay: A substrate solution was made by dissolving 1% Di-galactosyl-di-glyceride (with a purity >80%, possibly supplied by Sigma Aldrich or Avanti Polar Lipids), 6.5% (w/v) TRITON™-X 100 (Sigma X-100), and 5 mM CaCl₂) in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate (ThermoScientific #269620) and using a thermomixer (iEMS Incubator/shaker, Thermo Scientific). The amount of free fatty acid liberated during enzymation was measured using the NEFA kit (WakoChemicals GmbH, Germany, NEFA-HR(2) R1 #434-91795 and NEFA-HR(2) R2 #436-91995).

The amount of free fatty acid liberated during enzymation was measured using the NEFA kit and enzyme activity was calculated based on the use of an enzyme standard of SEQ ID NO: 1. The enzyme standard was assigned an activity of 3228 DGDG-U/g based on the use of an oleic acid standard.

### MGDG-U Assay:

Galactolipase activity (MGDG-U) was determined using the following assay: A substrate solution was made by dissolving 1% mono-galactosyl-di-glyceride (with a purity >80%, possibly supplied by Sigma Aldrich or Avanti Polar Lipids), 3.75% (w/v) TRITON™-X 100 (Sigma X-100), and 5 mM CaCl₂) in 0.05 M HEPES buffer pH 7.0. Enzyme samples and oleic acid standard (WakoChemicals GmbH, Germany, NEFA Standard, #270-77000) were diluted in 10 mM HEPES pH 7.0 containing 0.1% TRITON™ X-100. Enzyme samples were diluted to an appropriate concentration to achieve a linear response in the assay. Analysis was carried out in a 96 well micro titer plate (ThermoScientific #269620) and using a thermomixer (iEMS Incubator/shaker, Thermo Scientific). The amount of free fatty acid liberated during enzymation was measured using the NEFA kit (WakoChemicals GmbH, Germany, NEFA-HR(2) R1 #434-91795 and NEFA-HR(2) R2 #436-91995).

The amount of free fatty acid liberated during enzymation was measured using the NEFA kit and enzyme activity was calculated based on the use of an enzyme standard of SEQ ID NO: 1. The enzyme standard was assigned an activity of 16229 MGDG-U/g based on the use of an oleic acid standard.

It is to be understood that there is flexibility regarding the amount of TRITON™-X100 (Sigma X-100). 3.75% (w/v) TRITON may be used instead of 6.5% (w/v).

It is to be understood that the enzyme standard used in all the activity assays employing SEQ ID NO:1 is PowerBake 4080 available from IFF.

### Protein Determination by Stain Free Imager Criterion

Protein was quantified by SDS-PAGE gel and densitometry using GelDoc™ Go Imaging system (Bio-Rad). Reagents used in the assay: Concentrated (2×) Laemmli Sample Buffer (Bio-Rad, Catalogue #1610737); 26-well TGX Any kDa Gel (Bio-Rad, Catalogue #5678125); protein markers “Precision Plus Protein™ Unstained Protein Standards” (Bio-Rad, Catalogue #161-0363); protein standard based on SEQ ID NO: 1 (protein concentration assigned by Total Amino Acid Analysis, Eurofins Scientific). The analysis was carried out as follow: In a 96 well-PCR plate 50 μL diluted enzyme sample were mixed with 50 μL sample buffer containing 2.7 mg DTT. The plate was sealed by Microseal ‘B’ Film from Bio-Rad and placed into PCR machine and heated at 70° C. for 10 minutes. Next the chamber was filled with running buffer, gel cassette was set. Then 10 μL of each sample and standard (˜0.1-1.00 mg/mL SEQ ID NO:1) was loaded on the gel and 10 μL of the markers were loaded. After that the electrophoresis was run at 200 V for 34 min. Following electrophoresis the gel was transferred to the GelDoc Go Imager. Image Lab software was used for calculation of intensity of each band. By knowing the protein amount of the standard sample a calibration curve was created. The amount of target protein in the sample was determined by the band intensity and the calibration curve.

### Baking Applications

Crusty Roll baking setup

| Recipe | Bakers % |
|---|---|
| Wheat flour (Reform) | 100 |
| Compressed yeast (Malteserkors) | 4.5 |
| Salt | 1.6 |
| Sucrose | 1.6 |
| Water (400 BU-2%) | 59 |
| Fungal alpha amylase (16.2 FAU/g blend) | 0.47 |
| Other Enzymes | variable |

Kneading on a Diosna spiral mixer. Water uptake for flour according to analysis: 400 BU-2%

### Procedure

Mix all ingredients in a bowl, 1 minute slow speed—add water and knead 2 minutes slow and 6.5 minutes fast speed. Dough temperature must be approximate 26° C. 1350 g dough is scaled and molded round by hand. The dough is rested in a heating cabinet for 10 minutes at 30° C.

The dough is molded into 30 dough balls on a “GLIMIK™ rounder”—settings according to table on machine.

The dough is proofed for 45 minutes at 34° C., 85% RH and baked for 13 minutes at 200° C./2 1 steam+5 minutes damper open (MIWE oven prog. 1). After baking the rolls are cooled for 25 minutes at ambient temperature before weighing and measuring of volume.

Dough and bread characteristics are evaluated by a skilled person.

TABLE 2
Dough and Bread Characteristics and Evaluation

| Evaluation | Evaluation method | Lowest score = 1 | Highest score = 10 |
|---|---|---|---|
| Dough | | | |
| Dough | Extend dough with | Dough cannot be | Dough can be stretched |
| development | fingers | stretched without | obtaining papery thin |
| after mixing | | breaking | dough without breakage |
| Stickiness after | Cut a big slit in all | Dry surface. The | The dough sticks to |
| mixing | dough, open the dough, | dough slips your | your fingers |
| | touch the cut dough | fingers | |
| | surface with fingers | | |
| Extensibility | Extend dough with | Dough cannot be | Dough can be stretched |
| after resting | fingers | stretched without | obtaining papery thin |
| | | breaking | dough without breakage |
| Stickiness after | Cut a big slit in all | Dry surface. The | The dough sticks to |
| resting | dough, open the dough, | dough slips your | your fingers |
| | touch the cut dough | fingers | |
| | surface with fingers | | |
| Crust | | | |
| Crispiness of | Fracture crust using | Leathery crust | Crisp crust |
| crust | several fingers | | |
| Crumb | | | |
| Crumb pore size | Visual evaluation of | Open crumb, big gas | Fine crumb, small gas |
| | sliced bread, | bubbles | bubbles |
| | size of gas bubbles | | |
| | in crumb | | |
| Crumb pore | Visual evaluation of | Big variation in sizes | Constant gas bubble |
| homogeneity | sliced bread, | of gas bubbles | size |
| | homogeneity of gas | | |
| | bubbles | | |
| Product shape | | | |
| Capping/ | Visual evaluation of | A very large hole | No separation between |
| Hole under the | vertical cut surface | directly under the | crust and crumb. |
| crust | | crust. | |
| Oven | Visual evaluation | No energy | High level of energy |
| spring/Energy | amount of energy in the | | |
| | product | | |

### Example 1. Preparation of Lipases

Lipases of the present invention, having galactolipase activity and no and or low lysophospholipase activity can be wild type or engineered. Libraries of lipases can be screened for the appropriate properties according to the present invention. Alternatively, a lipase not having the desired properties can be modified via protein engineering to provide a lipase according to the present invention.

DNA manipulations to provide lipases of the present invention, i.e. lipases having galactolipase activity and no or low lyso-phospholipase activity, may be carried out using molecular biology techniques known in the art. Polynucleotide fragments corresponding to the coding sequences for the various lipases may be synthesized using preferred codons for the fungal expression host Trichoderma reseei (T. reesei) and assembled using PCR techniques. A suitable signal sequence e.g. the pep1 aspartate protease from T. reseei is introduced at the N-terminus (5′ end) of each lipase gene sequence. The Gateway® BP recombination technique is used to introduce the genes into the pDonor221 vector (Invitrogen, US) according to recommendations of the supplier. The resulting entry plasmids may be recombined with the destination vector pTTTpyr2 resulting in final expression vectors. pTTTpyr2 is similar to the pTTTpyrG vector described before (PCT publication WO 2011/063308), except that the pyrG gene is replaced with the pyr2 gene. Vector pTTTpyr2 contains the T. reesei cbhI promoter and terminator regions, the Aspergillus nidulans amdS selection marker, the T. reesei pyr2 selection marker, and telomeric sequences from T. reseei (for autonomous maintenance). These plasmids may be propagated in Escherichia coli TOP10 cells (Invitrogen, US), and the DNA is purified, and sequence verified.

All fungal manipulations, including high throughput transformations, inoculations, fermentations and harvesting may be performed robotically in either 24 or 96 well microtiter plates (MTP). Plasmids may be transformed into suitable T. reesei host strain using the polyethylene glycol (PEG)-protoplast method. In brief, transformation mixtures containing approximately 0.5-2 μg of DNA and 5×106 protoplasts in a total volume of 50 μL may be treated with 200 μL of 25% PEG solution followed by dilution with equal volume of 1.2 M sorbitol/10 mM Tris/10 mM CaCl2) pH 7.5 solution and poured in 24 well MTPs with 1 ml of 3% low melting agarose containing 1M sorbitol in minimal medium. After sufficient growth transformants from each well may be pooled together and plated on fresh 24 well agar MTPs. Once sporulated, spores may be harvested and used for inoculation of liquid cultures.

For the expression of lipase proteins, the transformed T. reseei strains may be cultured as follows: 10⁵-10⁶ T. reesei spores may be used to inoculate 1 ml of production medium (37 g/L glucose, 1.6 g/L sophorose, 9 g/L casamino acids, 10 g/L (NH4)2SO4, 4.5 g/L KH2PO4, 1 g/L MgSO4*7H2O, 1 g/L CaCl₂)*2H2O, 33 g/L PIPPS buffer (pH 5.5), 0.25% T. reesei trace elements (100%: 175 g/L citric acid (anhydrous), 200 g/L FeSO4*7H2O, 16 g/L ZnSO4*7H2O, 3.2 g/L CuSO4*5H2O, 1.4 g/L MnSO4*H2O, 0.8 g/L H3B03) in 24 well MTPs. MTPs may be incubated in a shaker incubator with a 50 mm throw at 200 rpm and 28 C with 80% humidity. After 5 days of fermentation, the cultures may be filtered by centrifugation using hydrophilic PVDF membranes to obtain clarified supernatants used for analysis of the recombinant lipase enzymes.

### Example 2. Purification and Quantification of Lipases

T. reseei strains encoding lipases of the present invention may be cultured as described above, and clarified supernatants may be used to determine the lipase substrate specificity. The lipases of the present invention may alternatively be further purified using the purification methods know in the art. The following method of purification may be used.

Clarified supernatants containing lipase may be concentrated and ammonium sulfate may be added to a final concentration of 1 M. The solution may be loaded onto a HiPrep™ Phenyl FF column pre-equilibrated with 20 mM NaAc (pH 5.0) supplemented with 1 M ammonium sulfate. The target protein may be eluted from the column with 0.1 M ammonium sulfate. The corresponding fractions may be pooled, concentrated and buffer-exchanged into 20 mM Tris (pH 7.5) (Buffer B) using a VivaFlow 200 ultra-filtration device (Sartorius Stedim). The resulting solution may be applied to a HiLoad™ Q FF 16/10 column pre-equilibrated with Buffer B. The target protein may be eluted from the column with 0.3 M NaCl. Fractions containing the active protein may be pooled, concentrated and buffer-exchanged into 20 mM NaAc (pH 5.0), 150 mM NaCl via the 10K Amicon Ultra filtration devices, and then stored in 40% glycerol at −20° C. until usage. Concentration of purified protein may be calculated by measuring the absorbance at 280 nm.

### Example 3. Activity Characterization of Lipases

Enzyme characterization is done by determination of specific activity using different lipid substrates as per activity methods presented in ‘Assays and Methods’. Powerbake 4080 is a commercial product of IFF. Powerbake 4080 is known to have both galactolipase and phospholipase activity. The active enzyme component of Powerbake 4080 is set forth as SEQ ID NO: 6 from U.S. Pat. No. 8,012,732 hereby incorporated by reference (also set forth herein as SEQ ID NO: 1). Lipopan F is a commercial product of Novozymes. The active enzyme in Lipopan F also known to have galactolipase activity and is in SEQ ID NO: 2 of EP0869167B hereby incorporated by reference (also set forth herein as SEQ ID NO: 2).

Lipases of the present invention may be characterized by their activity on 4 different substrates, PC, LPC, DGDG and MGDG using the assays mentioned under “Assays and Methods”. The results can be seen in table 4.

TABLE 3
Activity of lipases on PC, LPC, DGDG and MGDG substrates.

| | | | | | MGDG- |
|---|---|---|---|---|---|
| SEQ | DGDG- | LPC-U/DGDG-U | PC-U/DGDG-U | MGDG- | U/LPC-U |
| ID | U/mg | ratio | ratio | U/mg | ratio |
| 1 | 482 | 0.113 | 3.77 | 2422 | 45 |
| 2 | 1287 | 0.028 | 1.18 | 2276 | 64 |
| 3 | 23608 | 0.005 | 0.12 | 13652 | 110 |
| 4 | 18092 | 0.011 | 0.04 | 11561 | 59 |

### Example 4. Baking Experiments Testing Application Effect

In this experiment the candidates having low LPC-U/DGDG-U ratio were tested in a Crusty Roll experimental setup to show application performance.

The Crusty Roll baking was done according to ‘Crusty Roll’ description presented in the ‘Assay and Methods’ section above.

The experimental setup of the application trials and the results from the baking evaluation are presented in respectively.

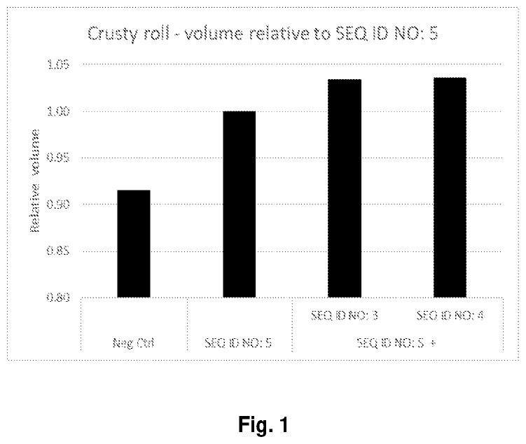

Table 4 and FIG. 1.

TABLE 4
Experimental setup evaluating LPC-U/DGDG-U candidates
in Crusty Roll, testing LPC-U/DGDG-U candidate
response baking on top of SEQ ID NO: 5

| | | | | SEQ ID | SEQ ID |
|---|---|---|---|---|---|
| | | | | NO: 5 + | NO: 5 + |
| | | Negative | SEQ ID | SEQ ID | SEQ ID |
| | | Control | NO: 5 | NO: 3 | NO: 4 |
| Dosage | SEQ ID NO: 5, | — | 8800 | 8800 | 8800 |
| U/kg | PC-U/kg flour | | | | |
| flour | LPC-U/DGDG-U | — | — | 100 | 100 |
| | candidate, DGDG- | | | | |
| | U/kg flour | | | | |

What is claimed is:

1. An isolated polypeptide comprising a lipase having galactolipase activity of at least 100 DGDG-U per mg of said lipase and having a ratio of LPC-U/DGDG-U below 0.02.

2. The isolated polypeptide of claim 1 having galactolipase activity of at least 150 DGDG-U per mg.

3. The isolated polypeptide of claim 2 having galactolipase activity of at least 200 DGDG-U per mg.

4. The isolated polypeptide of claim 3 having galactolipase activity of at least 250 DGDG-U per mg.

5. The isolated polypeptide of claim 4 having galactolipase activity of at least 300 DGDG-U per mg.

6. The isolated polypeptide of any of claims 1 to 5 having a ratio of LPC-U/DGDG-U below 0.01.

7. The isolated polypeptide of claim 6 having a ratio of LPC-U/DGDG-U below 0.009.

8. The isolated polypeptide of claim 7 having a ratio of LPC-U/DGDG-U below 0.007.

9. The isolated polypeptide of claim 8 having a ratio of LPC-U/DGDG-U below 0.005.

10. The isolated polypeptide of claim 9 having a ratio of LPC-U/DGDG-U below 0.003.

11. The isolated polypeptide of claim 10 having a ratio of LPC-U/DGDG-U below 0.001.

12. The isolated polypeptide of any of claims 1 to 11 further having a ratio of PC-U/DGDG-U above 0.01.

13. An isolated polypeptide comprising a lipase having a galactolipase activity of at least 100 MGDG-U per mg protein of said lipase and having a ratio of MGDG-U/LPC-U above 70, 75, 80, 85, 90, 95 or 100.

14. The isolated polypeptide of any of claims 1 to 13 wherein said lipase is an engineered lipase.

15. An isolated polynucleotide comprising a nucleic acid sequence encoding the isolated polypeptide of any one of claims 1 to 14.

16. A recombinant expression vector comprising a polynucleotide according to claim 15.

17. A host cell comprising the recombinant expression vector according to claim 16.

18. The host cell of claim 17 comprising a bacterial, fungal, yeast, plant or mammalian cell.

19. A method of making a dough, said method comprising admixing a dough component selected from the group consisting of flour, salt, water, sugar, fat, lecithin, oil and yeast with an isolated polypeptide according to any of claims 1 to 14.

20. The method of making a dough of claim 19 further comprising adding at least one additional enzyme useful for improving dough and/or a baked product made therefrom.

21. The method of making a dough of claim 20 wherein the additional enzyme is an amylase, cyclodextrin glucanotransferase, peptidase, transglutaminase, lipase other than said isolated polypeptide according to any of claims 1 to 14, phospholipase, galactolipase, cellulase, hemicellulase, protease, protein disulfide isomerase, glycosyltransferase, peroxidase, lipoxygenase, laccase, lactase, or oxidase.

22. The method of claim 21 wherein said amylase is an exoamylase.

23. The method of claim 22 wherein said exoamylase is a maltogenic amylase.

24. The method of claim 22 wherein said exoamylase is a non-maltogenic amylase.

25. The method of claim 24 wherein said non-maltogenic amylase hydrolyses starch by cleaving off one or more linear malto-oligosaccharides, predominantly comprising from four to eight D-glucopyranosyl units, from the non-reducing ends of the side chains of amylopectin.

26. The method of claim 20 wherein said additional enzyme is a phospholipase.

27. The method of claim 26 wherein the phospholipase comprises SEQ ID NO: 5 or an active fragment thereof.

28. The method of claim 27 wherein the active fragment is a mature polypeptide.

29. The method of any of claims 19 to 28 further comprising adding an emulsifier.

30. The method of claim 29 wherein the emulsifier is a phospholipid emulsifier.

31. The method of claim 30 wherein the phospholipid emulsifier is lecithin or lyso-lecithin.

32. The method of claim 29 wherein the emulsifier is a non-phospholipid emulsifier.

33. The method of claim 32 wherein the non-phospholipid emulsifier is DATEM, SSL, a monoglyceride or a diglyceride.

34. A pre-mix for baking comprising flour and an isolated polypeptide according to any of claims 1-14.

35. A pre-mix according to claim 34 further comprising at least one additional enzyme useful for improving dough and/or a baked product made therefrom.

36. A pre-mix according to claim 35 wherein the additional enzyme is an amylase, cyclodextrin glucanotransferase, peptidase, transglutaminase, lipase other than said isolated polypeptide according to any of claims 1 to 13, phospholipase, galactolipase, cellulase, hemicellulase, protease, protein disulfide isomerase, glycosyltransferase, peroxidase, lipoxygenase, laccase, lactase, or oxidase.

37. The pre-mix of claim 36 wherein said amylase is an exoamylase.

38. The pre-mix of claim 37 wherein said exoamylase is a maltogenic amylase.

39. The pre-mix of claim 37 wherein said exoamylase is a non-maltogenic amylase.

40. The pre-mix of claim 39 wherein said non-maltogenic amylase hydrolyses starch by cleaving off one or more linear malto-oligosaccharides, predominantly comprising from four to eight D-glucopyranosyl units, from the non-reducing ends of the side chains of amylopectin.

41. The pre-mix of claim 36 wherein said additional enzyme is a phospholipase.

42. The pre-mix of claim 41 wherein the phospholipase comprises SEQ ID NO: 5 or an active fragment thereof.

43. The pre-mix of claim 42 wherein the active fragment is a mature polypeptide.

44. A method of creating a lyso-galactolipid in a lipid containing food matrix comprising adding to the lipid containing food matrix an isolated polypeptide according to any of claims 1 to 14.

45. The method of creating a lyso-galactolipid in a lipid containing food matrix of claim 44 wherein said lipid containing food matrix is selected from the group consisting of eggs and food products containing eggs, dough for sweet bakery goods, processed meat, milk based products, and vegetable oil.

46. A method of creating a lyso-galactolipid in a lipid containing animal feed matrix or a grain based matrix comprising adding to the lipid containing animal feed matrix or the grain based matrix an isolated polypeptide according to any of claims 1 to 14.

47. A dough comprising an isolated polypeptide according to any of claims 1-14.

48. The dough of claim 47 having improved dough extensibility and/or stability.

49. The dough of claim 47 or 48 further comprising at least one additional enzyme useful for improving dough and/or a baked product made therefrom.

50. The dough of claim 49 wherein the additional enzyme is an amylase, cyclodextrin glucanotransferase, peptidase, transglutaminase, lipase other than said isolated polypeptide according to any of claims 1 to 13, phospholipase, galactolipase, cellulase, hemicellulase, protease, protein disulfide isomerase, glycosyltransferase, peroxidase, lipoxygenase, laccase, lactase, or oxidase.

51. The dough of claim 50 wherein the additional enzyme is an amylase.

52. The dough of claim 51 wherein said amylase is an exoamylase.

53. The dough of claim 52 wherein said exoamylase is a maltogenic amylase.

54. The dough of claim 52 wherein said exoamylase is a non-maltogenic amylase.

55. The dough of claim 54 wherein said non-maltogenic amylase hydrolyses starch by cleaving off one or more linear malto-oligosaccharides, predominantly comprising from four to eight D-glucopyranosyl units, from the non-reducing ends of the side chains of amylopectin.

56. The dough of claim 50 wherein said additional enzyme is a phospholipase.

57. The dough of claim 56 wherein the phospholipase comprises SEQ ID NO: 5 or an active fragment thereof.

58. The dough of claim 57 wherein the active fragment is a mature polypeptide.

59. A method of preparing a baked product comprising baking a dough according to any of claims 47 to 58.

60. A baked product according to claim 59.

61. The baked product of claim 60 having at least one improved property selected from the group consisting of improved crumb pore size, improved uniformity of gas bubbles, no separation between crust and crumb, increased volume, increased crust crispiness and improved oven spring.

62. An isolated polypeptide comprising a lipase having at least 75, 80, 85, 90, 95, 98, 99 or 100% sequence identity to SEQ ID NO:3 or SEQ ID NO:4 or a lipase active fragment thereof.

63. The isolated polypeptide of claim 62 wherein the lipase active fragment comprises a mature polypeptide.
